(19) United States
(12) Patent Application Publication
(10) Pub. No.: US 2021/0259728 A1
(43) Pub. Date: Aug. 26, 2021

(54) ADJUSTABLE LENGTH TROCAR

(71) Applicant: Covidien LP, Mansfield, MA (US)

(72) Inventors: Justin J. Thomas, New Haven, CT (US); Jacob C. Baril, Norwalk, CT (US); Matthew A. Dinino, Newington, CT (US); Roy J. Pilletere, North Haven, CT (US); Eric L. Brown, Haddam, CT (US); Nicolette R. LaPierre, Windsor Locks, CT (US); Gregory R. Morck, Haddam, CT (US)

(21) Appl. No.: 16/799,908

(22) Filed: Feb. 25, 2020

(51) Int. Cl. A61B 17/34; A61B 17/00

(52) U.S. Cl. CPC A61B 17/3417; A61B 17/00234; A61B 2017/00238; A61B 2017/3443; A61B 2017/347; A61B 2017/349

(57) ABSTRACT

A trocar is provided for providing access to body cavities during minimally invasive surgeries. The trocar possesses an elongate body formed of an outer tube and an inner tube, both of which are threaded. Rotating the outer tube about the inner tube permits adjustment of the length of the elongate body of the trocar. A locking collar on the trocar is used to maintain the elongate body at the desired length.

### TECHNICAL FIELD

The disclosure relates to a surgical apparatus for use in minimally invasive surgical procedures, such as endoscopic and/or laparoscopic procedures and, more particularly, to a trocar that may be adjusted to a proper length prior to use.

### BACKGROUND

Minimally invasive surgery, such as endoscopic surgery, reduces the invasiveness of surgical procedures. Endoscopic surgery involves surgery through body walls, for example, viewing and/or operating on the ovaries, uterus, gall bladder, bowels, kidneys, appendix, etc. There are many common endoscopic surgical procedures, including arthroscopy, laparoscopy, gastroentroscopy and laryngobronchoscopy, just to name a few. In these procedures, trocars are utilized for creating incisions through which the endoscopic surgery is performed. Trocar tubes or cannula devices are extended into and left in place in the abdominal wall to provide access for endoscopic surgical tools. A camera or endoscope is inserted through a trocar tube to permit the visual inspection and magnification of the body cavity. The surgeon can then perform diagnostic and/or therapeutic procedures at the surgical site with the aid of specialized instrumentation, such as forceps, graspers, cutters, applicators, and the like, which are designed to fit through additional cannulas.

Improved trocars for accessing a patient's body cavity remain desirable.

### SUMMARY

Trocars for use in minimally invasive surgery are provided. In aspects, the disclosed trocar includes an elongate body having a proximal portion and a distal portion, the elongate body formed of an inner tube and an outer tube; a seal assembly at the proximal portion of the elongate body; and a locking collar at the proximal portion of the elongate body. The inner tube moves within the outer tube to adjust a length of the elongate body.

In aspects, the inner tube of the elongate body has an outer surface possessing a threaded configuration. The outer tube of the elongate body also has an inner surface possessing a threaded configuration. In some aspects, the threaded configuration of the outer tube is female threading, and the threaded configuration of the inner tube is male threading.

In aspects, the length of the elongate body is adjusted by rotating the outer tube about the inner tube.

In aspects, the locking collar is closed when the elongate body is a desired length.

In some aspects, the locking collar is present at a proximal portion of the outer tube.

In aspects, the locking collar possesses a circular body and a C-shaped body joined by a pin.

The circular body of the locking collar can be adhered to the proximal portion of the outer tube by adhesives, soldering, riveting, or combinations thereof.

In some aspects, the circular body of the locking collar is riveted to the proximal portion of the outer tube.

In aspects, the proximal portion of the outer tube has a gap therein, which permits the proximal portion of the outer tube to flex and compress the inner tube when the locking collar is closed.

In aspects, a trocar of the disclosure has an elongate body having a proximal portion and a distal portion, the elongate body formed of an inner tube possessing a threaded configuration and an outer tube possessing a threaded configuration; a seal assembly at the proximal portion of the elongate body; and a locking collar at the proximal portion of the elongate body. The inner tube moves within the outer tube to adjust the length of the elongate body, and the locking collar is closed when the elongate body is a desired length.

### BRIEF DESCRIPTION OF THE DRAWINGS

Various aspects of the disclosed trocar are described herein below with reference to the drawings, wherein:

FIG. 1 is a side view of a trocar of the disclosure, showing a locking collar capable of fixing a length of the body of the trocar;

FIG. 2 is an exploded side view of an inner tube, an outer tube, and the locking collar used to form a body of the trocar of the disclosure;

FIG. 3 is a side perspective view showing adjustment of the length of the body of the trocar of FIG. 1, by rotating the inner tube within the outer tube;

FIG. 4 is a cross-sectional view of the locking collar in an open position shown along section line 4-4 in FIG. 3;

FIG. 5 is a is a cross-sectional view of the locking collar in a closed position shown along section line 5-5 in FIG. 1; and

FIG. 6 is a side view of the trocar of the disclosure after adjustment of body length, prior to use in a patient.

### DETAILED DESCRIPTION

The disclosed trocars are described in detail with reference to the drawings, in which like reference numerals designate identical or corresponding elements in each of the several views. As used herein the term “distal” refers to that portion of the trocar, or component thereof, farther from the user, while the term “proximal” refers to that portion of the trocar, or component thereof, closer to the user.

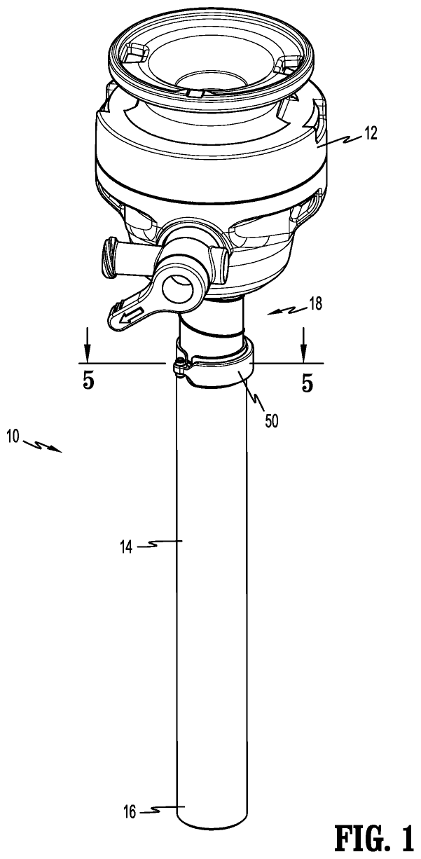

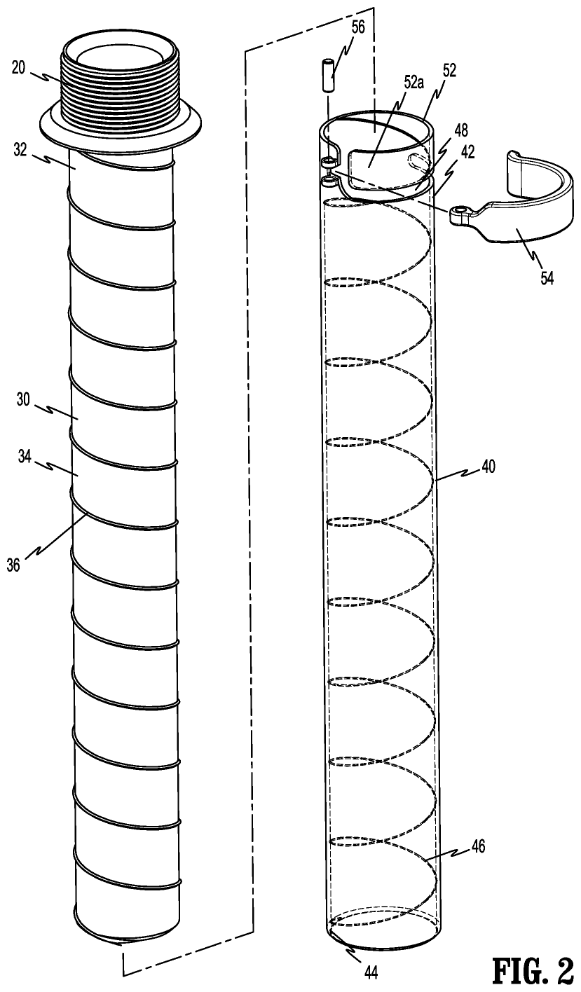

With reference to FIGS. 1-2, a trocar in accordance with an aspect of the disclosure is shown generally as trocar 10 that includes a seal assembly 12, an elongate body 14, and a distal portion 16. The seal assembly 12 may be attached to the elongate body 14 by threads 20 (FIG. 2) on a proximal portion 18 of the elongate body 14. The elongate body 14 also possesses a locking collar 50 at the proximal portion 18 of the elongate body 14.

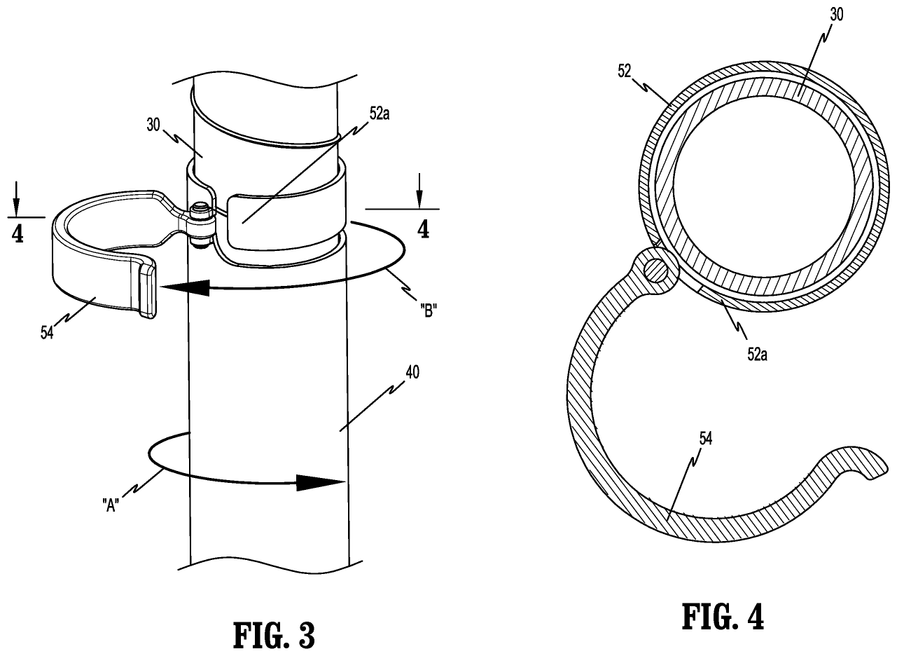

As shown in greater detail in FIGS. 2-3, elongate body 14 is formed of an inner tube 30 and an outer tube 40. Inner tube 30 moves within outer tube 40 between a generally retracted position (FIG. 1) to a generally extended position (FIG. 6).

In aspects, inner tube 30 also has an outer surface 34 possessing a threaded configuration 36. Outer tube 40 has an inner surface 44 possessing a threaded configuration 46 (shown in phantom in FIG. 2), which interlocks with the threaded configuration 36 on the outer surface 34 of the inner tube 30.

The threaded configuration 46 on the inner surface 44 of the outer tube 40 and the threaded configuration 36 on the outer surface 34 of the inner tube 30 allows the end user to adjust the length of the elongate body 14 by rotating the outer tube 40 about the inner tube 30, as indicated by arrow “A” in FIG. 3. Rotating the outer tube 40 about the inner tube 30 in the direction indicated by the arrow “A” will increase the length of the elongate body 14 by extending the inner tube 30 out of a proximal portion 42 of the outer tube 40. To shorten the length of the elongate body 14, the end user would rotate the outer tube 40 about the inner tube 30 in a direction opposite to the direction indicated by arrow “A” (now shown).

In aspects, the threaded configuration 46 of the outer tube 40 may be female threading, while the threaded configuration 36 of the inner tube 30 may be male threading. In other aspects, the threading configurations may be reversed, i.e., the threaded configuration 46 of the outer tube 40 may be male threading, while the threaded configuration 36 of the inner tube 30 may be female threading. The male threads engage with the female threads, allowing for movement of the outer tube 40 about the inner tube 30 and thereby allowing the end user to adjust the length of the elongate body 14.

While the above disclosure has discussed threading of the inner tube 30 and the outer tube 40 for adjusting the length of the elongate body 14, other mechanisms for adjusting the length of the elongate body 14, e.g., a telescoping configuration (not shown) where the inner tube 30 extends from the outer tube 40, are also contemplated.

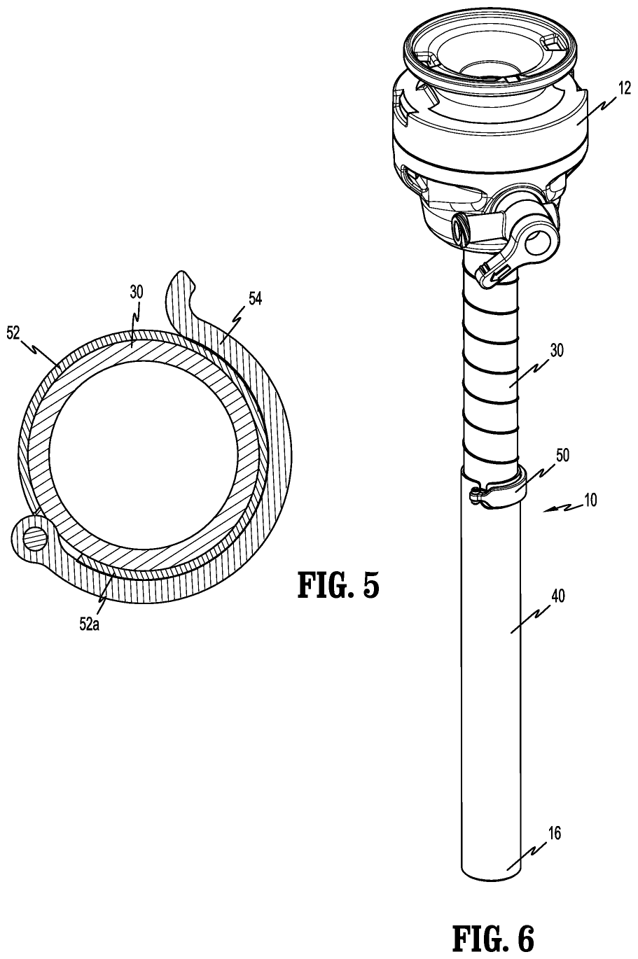

The locking collar 50 is used to fix the elongate body 14 at a desired length. As shown in FIG. 2, the locking collar 50 is present at a proximal portion 42 of the outer tube 40. The locking collar 50 possesses a circular body 52 and a C-shaped body 54 joined to the circular body 52 by a pin 56. The circular body 52 includes a flexible arm 52a that is secured to the proximal portion 42 of the outer tube 40 in cantilevered fashion. The circular body 52 is not a complete circle. When the C-shaped body 54 is in a non-deployed state (FIGS. 3 and 4), it is pivoted away from the outer tube 40, as indicated by arrow “B” in FIG. 3. Once the desired length of the elongate body 14 has been obtained, the C-shaped body 54 of the locking collar 50 is pressed to pivot the C-shaped body 54 so it is in its deployed state, positioned about the circular body 52 of the locking collar 50 (FIG. 5). This is referred to, in aspects, as closing the locking collar 50. Closing the locking collar 50 so the C-shaped body 54 is in its deployed state results in compression of the flexible arm 52a of the circular body 52 about the outer tube 40 (FIGS. 5 and 6), thereby supplying pressure to the elongate body 14 so that the inner tube 30 may not be rotated within the outer tube 40 and the length of the elongate body 14 may not be adjusted any further.

The circular body 52 of the locking collar 50 may be integrally formed with the proximal portion 42 of the outer tube 40. Alternately, the circular body 52 can be adhered to the proximal portion 42 of the outer tube 40 by any suitable means, including adhesives, soldering, riveting, combinations thereof, and the like. In aspects, the circular body 52 of the locking collar 50 is riveted to the proximal portion 42 of the outer tube 40. As shown in FIG. 2, in aspects, gap 48 is defined between the proximal portion 42 of the outer tube 40 and the flexible arm 52a of the circular body 50. This gap 48 permits the flexible arm 52a of the locking collar 50 to flex and be compressed onto the inner tube 30, thereby supplying pressure to the elongate body 14 so that the inner tube 30 may not be rotated within the outer tube 40 and the length of the elongate body 14 may not be adjusted any further.

Of course, if the length of the elongate body 14 needs further adjustment, the locking collar 50 may be opened to the non-deployed state (FIG. 4), the outer tube 40 may be rotated about the inner tube 30 to arrive at the desired length of the elongate body 14 (FIG. 3), and the C-shaped body 54 of locking collar 50 may then be pressed adjacent the circular body 52 to close the locking collar 50 and maintain the desired length of the elongate body 14 of the trocar 10.

As shown in FIG. 6, once the desired length of the elongate body 14 has been obtained by rotating the outer tube 40 about the inner tube 30, the locking collar 50 is closed, thereby fixing the desired length of the elongate body 14.

Once the trocar 10 has been adjusted to the desired length, the trocar 10 is ready for use in a patient as part of an intended procedure.

Any of the components described herein may be fabricated from either metals, plastics, resins, composites or the like taking into consideration strength, durability, wearability, weight, resistance to corrosion, ease of manufacturing, cost of manufacturing, and the like. In aspects, the inner tube and the outer tube of the elongate body of the trocar may be made of metals, such as stainless steel, while the locking collar may be made of a resin, such as a thermoplastic resin.

It will be understood that various modifications may be made to the disclosed trocars. Therefore, the above description should not be construed as limiting, but merely as exemplifications of aspects of the disclosure. Those skilled in the art will envision other modifications within the scope and spirit of the disclosure. For example, any and all features of one described aspect may be suitably incorporated into another aspect.

What is claimed is:

1. A trocar, comprising:
an elongate body having a proximal portion and a distal portion, the elongate body formed of an inner tube and an outer tube;
a seal assembly at the proximal portion of the elongate body; and
a locking collar at the proximal portion of the elongate body,
wherein the inner tube moves within the outer tube to adjust a length of the elongate body.

2. The trocar of claim 1, wherein the inner tube of the elongate body has an outer surface possessing a threaded configuration, and the outer tube of the elongate body has an inner surface possessing a threaded configuration.

3. The trocar of claim 2, wherein the threaded configuration of the outer tube is female threading, and the threaded configuration of the inner tube is male threading.

4. The trocar of claim 2, wherein the length of the elongate body is adjusted by rotating the outer tube about the inner tube.

5. The trocar of claim 1, wherein the locking collar is closed when the elongate body is a desired length.

6. The trocar of claim 1, wherein the locking collar is present at a proximal portion of the outer tube.

7. The trocar of claim 1, wherein the locking collar possesses a circular body and a C-shaped body joined by a pin.

8. The trocar of claim 1, wherein the circular body of the locking collar is adhered to the proximal portion of the outer tube by adhesives, soldering, riveting, or combinations thereof.

9. The trocar of claim 8, wherein the circular body of the locking collar is riveted to the proximal portion of the outer tube.

10. The trocar of claim 1, wherein the proximal portion of the outer tube has a gap therein, which permits the proximal portion of the outer tube to flex and compress the inner tube when the locking collar is closed.

11. A trocar, comprising:
an elongate body having a proximal portion and a distal portion, the elongate body formed of an inner tube possessing a threaded configuration and an outer tube possessing a threaded configuration;
a seal assembly at the proximal portion of the elongate body; and
a locking collar at the proximal portion of the elongate body,
wherein the inner tube moves within the outer tube to adjust the length of the elongate body, and
wherein the locking collar is closed when the elongate body is a desired length.

12. The trocar of claim 11, wherein the threaded configuration of the outer tube is female threading, and the threaded configuration of the inner tube is male threading.

13. The trocar of claim 11, wherein the length of the elongate body is adjusted by rotating the outer tube about the inner tube.

14. The trocar of claim 11, wherein the locking collar is present at a proximal portion of the outer tube.

15. The trocar of claim 11, wherein the locking collar possesses a circular body and a C-shaped body joined by a pin.

16. The trocar of claim 11, wherein the circular body of the locking collar is adhered to the proximal portion of the outer tube by adhesives, soldering, riveting, or combinations thereof.

17. The trocar of claim 16, wherein the circular body of the locking collar is riveted to the proximal portion of the outer tube.

18. The trocar of claim 11, wherein the proximal portion of the outer tube has a gap therein, which permits the proximal portion of the outer tube to flex and compress the inner tube when the locking collar is closed.
